(19) United States
(12) Patent Application Publication
(10) Pub. No.: US 2012/0149076 A1
(43) Pub. Date: Jun. 14, 2012

(54) INTEGRATION OF FERMENTAITON WITH MEMBRANE

(75) Inventor: Cesar B. Granda, College Station, TX (US)

(73) Assignee: TERRABON MIX-ALCO, INC., Houston, TX (US)

(21) Appl. No.: 13/311,533

(22) Filed: Dec. 5, 2011

Related U.S. Application Data
(60) Provisional application No. 61/420,412, filed on Dec. 7, 2010.

(51) Int. Cl. C12P 1/00; C12P 5/00; C12P 7/62; C12P 7/02; C12P 7/40; C12P 7/26

(52) U.S. Cl. 435/135; 435/41; 435/136; 435/148; 435/155; 435/166

(57) ABSTRACT

Herein disclosed is a method comprising a) fermenting biomass to produce a fermentation broth; b) separating the fermentation broth into a liquid stream and a solid or slurry stream; c) passing the liquid stream through a reverse osmosis membrane to obtain a permeate and a retentate; and d) concentrating the retentate. Herein disclosed is a method comprising a) fermenting biomass in a first fermentor to produce a first fermentation broth; b) separating the first fermentation broth into a first liquid stream and a first solid or slurry stream; c) introducing the first solid or slurry stream into a second fermentor to produce a second fermentation broth, wherein the second fermentor comprises a lower fermentation products concentration than the first fermentor; d) separating the second fermentation broth into a second liquid stream and a second solid or slurry stream; and e) passing the second liquid stream through a reverse osmosis membrane.

### CROSS-REFERENCE TO RELATED APPLICATIONS

This application claims the benefit under 35 U.S.C. §119(e) of U.S. Provisional Patent Application No. 61/420,412 filed Dec. 7, 2010, the disclosure of which is hereby incorporated herein by reference.

### STATEMENT REGARDING FEDERALLY SPONSORED RESEARCH OR DEVELOPMENT

Not applicable.

### BACKGROUND

This disclosure relates generally to the conversion of biomass to products. More specifically, this disclosure describes processes and systems to convert solid biomass to fuels.

### SUMMARY

Herein disclosed is a method comprising a) fermenting biomass to produce a fermentation broth; b) separating the fermentation broth into a liquid stream and a solid or slurry stream; c) passing the liquid stream through a reverse osmosis membrane to obtain a permeate and a retentate; and d) concentrating the retentate to produce a concentrate. In some embodiments, Step d comprises evaporating the retentate to produce a condensate in addition to the concentrate. In some embodiments, Step b comprises utilizing a separation system selected from the group consisting of a high-speed centrifuge, a flocculation/coagulation system, a fine filtration system, a microfiltration membrane system, an ultrafiltration membrane system, a nanofiltration membrane system, and combinations thereof.

In some embodiments, Step a further comprises producing a gas stream and recovering a gas product from the gas stream. In some embodiments, Step a further comprises fermenting the biomass in a fermentor to produce a gas stream; and introducing the gas stream to a bioreactor wherein autotrophic conversion takes places and wherein the fermentor is configured to receive a product stream from the bioreactor. In some embodiments, the gas stream comprises carbon dioxide and hydrogen. In some embodiments, the reverse osmosis membrane comprises ceramic membranes. In some embodiments, the reverse osmosis membrane comprises an anti-fouling mechanism. In some embodiments, the anti-fouling mechanism comprises the application of electric fields.

In some embodiments, the reverse osmosis membrane is selected from the group consisting of a composite of ceramic membrane with silver, a composite of ceramic membrane with titanium, a composite of ceramic membrane with alumina; a nano-composite material containing silver, and a nano-composite material containing silica.

In some embodiments, the method further comprises recycling the permeate and the condensate to Step a. In some embodiments, the method further comprises processing the concentrate to produce a product, wherein product is selected from the group consisting of salts, acids, ketones, esters, alcohols, and hydrocarbons.

Herein also disclosed is a method comprising a) fermenting biomass in a first fermentor to produce a first fermentation broth; b) separating the first fermentation broth into a first liquid stream and a first solid or slurry stream; c) introducing the first solid or slurry stream into a second fermentor to produce a second fermentation broth, wherein the second fermentor comprises a lower fermentation products concentration than the first fermentor; d) separating the second fermentation broth into a second liquid stream and a second solid or slurry stream; and e) passing the second liquid stream through a reverse osmosis membrane to obtain a permeate and a retentate.

In some embodiments, the method further comprises concentrating the first liquid stream or concentrating the first liquid stream and the retentate. In some embodiments, concentrating comprises evaporation to produce a condensate and a concentrate. In some embodiments, the method further comprises recycling the permeate and the condensate to Step a. In some embodiments, the method further comprises recycling the permeate and the condensate to Step c. In some embodiments, at least a portion of the second liquid stream is sent to Step a. In some embodiments, the retentate is sent to Step a. In some embodiments, Step b or Step d comprises utilizing a separation system selected from the group consisting of a high-speed centrifuge, a flocculation/coagulation system, a fine filtration system, a microfiltration membrane system, an ultrafiltration membrane system, a nanofiltration membrane system, and combinations thereof.

Herein also disclosed is a system comprising a fermentor configured to receive biomass and to produce a liquid/solid slurry; a separator configured to receive the liquid/solid slurry from the fermentor and to produce a liquid stream and a solid or slurry stream; a reverse osmosis membrane configured to receive the liquid stream from the separator and to produce a retentate and a permeate; and an evaporator configured to receive the retentate and to produce a concentrate and a condensate. In some embodiments, the system further comprises a bioreactor coupled with the fermentor, wherein the bioreactor is configured to receive a gas stream from the fermentor and the fermentor is configured to receive a product stream from the bioreactor.

Herein also disclosed is a system comprising a first fermentor configured to receive biomass and to produce a first liquid/solid slurry; a first separator configured to receive the first liquid/solid slurry from the first fermentor and to produce a first liquid stream and a first solid or slurry stream; a second fermentor configured to receive the first solid stream and to produce a second liquid/solid slurry, wherein the second fermentor is operated at a lower fermentation products concentration than the first fermentor; a second separator configured to receive the second liquid/solid slurry from the second fermentor and to produce a second liquid stream and a second solid or slurry stream; and a reverse osmosis membrane configured to receive the second liquid stream from the second separator and to produce a retentate and a permeate. In some embodiments, the system further comprises an evaporator configured to receive the first liquid stream or the first liquid stream and the retentate and to produce a concentrate and a condensate.

These and other embodiments, features and advantages will be apparent in the following detailed description and drawings.

### BRIEF DESCRIPTION OF THE DRAWINGS

FIG. 1 is an illustration of cost response curve as a function of the target final product concentration.

FIG. 2 is an illustration of an adequate value proposition to follow in concentrating a solution, where the thicker line minimizes cost.

FIG. 3 illustrates an integrated process of a low-product-concentration fermentation with reverse osmosis (RO) followed by evaporation, according to an embodiment of this disclosure.

FIG. 4 illustrates an integrated process of a low-product-concentration fermentation with reverse osmosis (RO) followed by evaporation with optional recovery of H₂ produced during fermentation by means of autotrophic conversion of H₂ and CO₂ into a product, according to an embodiment of this disclosure.

FIG. 5 illustrates an integrated process of a low-product-concentration fermentation and a high-product-concentration fermentation with reverse osmosis (RO) followed by evaporation, wherein the clean water is shared between the two fermentations, according to an embodiment of this disclosure.

FIG. 6 illustrates an integrated process of a low-product-concentration fermentation and a high-product-concentration fermentation with reverse osmosis (RO) followed by evaporation, wherein some of the low-product-concentration liquid is used as the liquid input for the high-product-concentration fermentation, according to an embodiment of this disclosure.

FIG. 7 illustrates an integrated process of a low-product-concentration fermentation and a high-product-concentration fermentation with reverse osmosis (RO) followed by evaporation, wherein the retentate out of the RO system is used as the liquid input for the high-product-concentration fermentation, according to an embodiment of this disclosure.

### DETAILED DESCRIPTION

The conversion of biomass to biofuels has many benefits, such as reducing dependence on foreign oil, reducing trade deficits, improving rural economies, and addressing man-made climate change. Although there are many technologies, most efforts have focused on using fermentation for converting biomass into biofuels such alcohols (e.g., ethanol, butanol), or making intermediate products that can then be converted to fuels, such as acetone and organic acids (e.g., acetic acid, butyric acid).

In most fermentation technologies, product inhibition represents a significant barrier to high conversion, high yields and high product concentration. Many efforts have been considered for integration of product formation and recovery to remove product inhibition. If it was possible to do away with or at least minimize the need for attaining high product concentrations, it has been shown that the fermentation may be run at such conditions that would allow for very high yields (which translates into higher revenue), very high feedstock loading rate (which translates into lower capital costs due to smaller fermentors) and low suspended solids concentration within the fermentor or bioreactor when converting solid biomass (which translates into lower capital and operating expenses in solids handling).

In fermentations, low product concentrations to attain low inhibitions are attained by low Liquid Residence Times (LRT); however, most of the time, because liquid and substrate are fed together and removed together from the system, LRT is coupled to the substrate or Feedstock Residence Time in the system (FRT). ERT, on the contrary, should be maximized to obtain high conversions and high yields. Decoupling FRT from LRT is very important. Such task is difficult when the feedstock is soluble (e.g., free sugars), but it is not as critical compared to when the feedstock is a solid because typically soluble feedstocks are much more easily consumed and converted by microorganisms. With solid feedstocks, liquid/solid separators such as filters, centrifuges, or screw presses; may be employed to separate the liquid from the solids. The liquid is then allowed to exit the fermentation, while most of the solids are retained and/or recycled to the fermentation. Given that the least expensive, most widely available biomass is solid (i.e., lignocellulose), this is the focus of this disclosure.

Although low product concentrations are easily attained, the issue is that attaining an economically feasible product recovery becomes a challenge because of the large quantities of water that must be removed. Attaining such economic recovery in spite of the low product concentration is therefore needed.

Efficient water-removal/dewatering techniques. There are several, well-known water removal (dewatering) techniques. The following are a few:

(1) Membrane dewatering systems employ semi-permeable membranes to filter out particles and even molecules of different sizes. Depending on the size of the pore, membrane filtration is known, from larger to smaller pore, as microfiltration, ultrafiltration, nanofiltration and reverse osmosis (RO). RO is typically used to allow water molecules to pass through, while retaining bigger molecules and thus it is a dewatering technique. The liquid feed enters the membrane system and it is divided into two streams: 1) the permeate, which is the part of the liquid that went through the membrane, and 2) the retentate, which is the more concentrated part that did not go through the membrane and exits the system.

(2) Mechanical dewatering may make use of evaporators, which may be of many types (e.g., forced-recirculation, falling film, flash) to remove water from a solution containing preferably non-volatile compounds, such as when recovering organic salts of acetic acid or lactic acid. The evaporators can have different arrangements, such as multiple effect evaporators, multi-stage flash evaporators and vapor-recompression evaporation. The evaporators may be driven by steam or they may be driven, as in the case of mechanical vapor recompression, by a compressing device such as a blower or a compressor. The liquid feed enters the evaporator, and the water is evaporated leaving behind the concentrated liquid, known as concentrate. If the evaporated water is condensed, this condensate may be recycled or exported.

(3) Distillation is typically used when the product to be removed is more volatile than water, such as for ethanol distillation. In such cases, it is the product that is removed from the water by evaporation as opposed to the water from the fermentation product. For a more efficient separation, a column with trays or packing might be employed. The lighter component exits the top of the column as the distillate, while the heavier components exit the bottom of the columns as the bottoms.

(4) Liquid-liquid extraction is another technique that, similar to distillation, may remove the product from the fermentation solution, leaving behind the water, although there are some liquid-liquid extraction techniques that do remove the water from the product. This extraction is done by contacting an extracting solvent, also known as extractant, with the solution containing the product of interest. The extracting solvent has affinity for the component that is being extracted from the solution (either the product or the water) and it can be later separated from this component more efficiently than separating the water from the product. The residual solution left behind after the component has been extracted is known as raffinate and the extractant phase containing the extracted component is known as extract. The most significant concern with this technique is extractant losses with the water stream.

Each of the techniques described above and many others used in separating water from a fermentation product has its own unique advantages and shortcomings. Depending on the needs of a particular process, one technique should be used over the others and many times a single dewatering approach that satisfies all needs is not necessarily efficient or even feasible. It is sometimes advantageous to combine two or more of these techniques in an effort to make a process feasible and/or more efficient.

In the present disclosure, RO membrane separation is utilized, followed by evaporation, to handle the low product concentrations that may be obtained in biomass fermentation, whose products have little or no volatility, such as carboxylic-acid salts (e.g., acetate, propionate, and butyrate). The low product concentration facilitates and enhances the fermentation of biomass.

Integration of RO and evaporation. The combination of RO and evaporation is able to efficiently dewater dilute broth from biomass fermentation that generates low-volatility or non-volatile products.

In RO membrane processes, the flux, that is, the flow of water through the membrane per unit of surface area, is expressed as follows:

$$J = \frac{1}{{R}_{m} + {R}_{n} + …} \cdot ( Δ P - ΔΠ )$$

Where,

J=Flux

Rₘ=Membrane resistance, which is inversely proportional, to permeability

R_{n, . . .} =Resistance due to other causes, such as fouling and compaction

ΔP=Pressure difference between the feed and the permeate

ΔΠ . . . =Osmotic pressure

The osmotic pressure in turn can be approximate as

Π=vnₛRT

where,

vnₛ=Total concentration of ions

R=Universal gas constant

T=Temperature

It is clear from the equations above that as long as the product concentration of the retentate is kept low, RO is an efficient dewatering technique, but as the product concentration increases in the feed or final retentate, the osmotic pressure will increase; therefore, the pressure of the feed needs to be increased to maintain an adequate flux through the membrane. As this occurs, costs increase and membrane problems, such as fouling, become more severe.

In addition to the high osmotic pressure, it is also important to make sure that the membranes are easily maintained. For this purpose, the use of ceramic membranes is recommended because they are more durable and easier to clean. In addition, because this system is employed as part of a process that employs fermentation, microbial growth is a concern in membranes because it causes fouling. Several techniques are being developed and may be used to inhibit microbial growth and reduce fouling on membranes such as composites of ceramic membranes with silver, titanium, alumina and others, nano-composite material containing certain elements such as silver and silica, and the application of electric fields.

On the other end of the spectrum, evaporation makes use of steam and mechanical devices, such as pumps, compressors and heat exchangers to remove water. The amount of steam and the size of mechanical devices, as well as the energy required for its operation, require a heavier capital investment and operating costs which increase in direct proportion to the amount of water that needs to be removed.

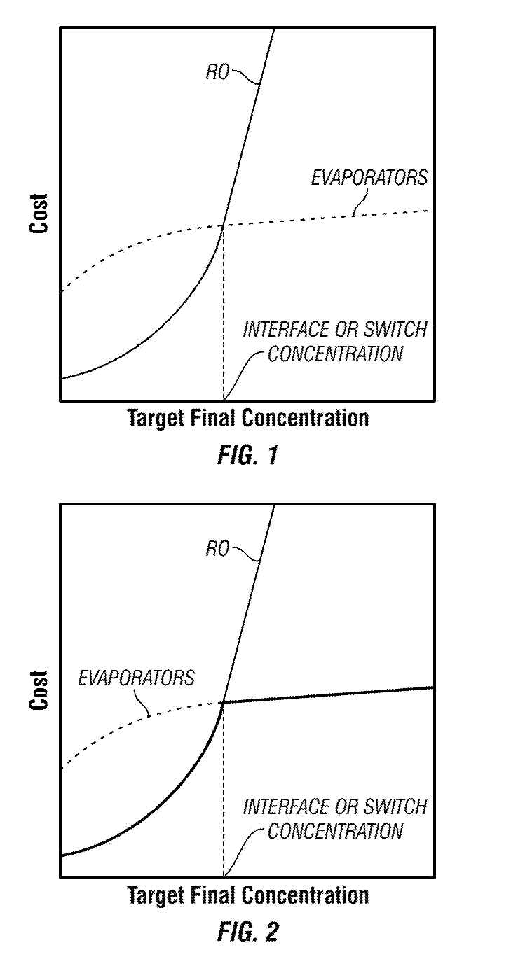

At low initial and final concentrations of the solution to be dewatered, where the osmotic pressure is low, typically evaporators cannot compete with RO. Conversely, high concentrations of the initial and final product do not affect the performance of evaporators significantly; therefore, RO cannot compete with evaporators at high concentrations. This behavior is illustrated in FIG. 1. There exists an optimal final target concentration, known as “switch” or “interface” concentration, at which there is a shift of cost efficiencies from RO to evaporation. At final target concentrations lower than the switch concentration, it is more economical to use RO for dewatering a solution. At final target concentrations higher than the switch concentration, it would be more economical to use evaporators for dewatering.

A good value proposition would be to follow the cost curves as illustrated by the thicker line in FIG. 2, where RO is employed to raise the concentration of the solution in question up to the switch concentration, and then evaporation is used to bring the solution from the switch concentration to the final desired target.

Application in Fermentation. An example of a fermentation where low-volatility or non-volatile products are produced is biomass fermentation for making organic acids (e.g., acetic, propionic, butyric acid). In this fermentation, the produced acids must be neutralized with a buffering agent (e.g., sodium carbonate, magnesium carbonate or oxide, calcium carbonate, ammonium bicarbonate) thus producing salts of the acids (e.g., acetate, propionate, butyrate), which are virtually non-volatile. These salts exit the fermentation in a high-water-content broth that must be dewatered before continuing downstream for further processing.

Some attempts have been made to remove product inhibition in these fermentations with perextraction (membrane-aided extraction), or with simple extraction during fermentation (extractive fermentation), however the effort was directed to the removal of the product, rather than to a dilute fermentation that may be economically dewatered as in this case. The integration of dilute fermentation with RO followed by evaporation for dewatering the fermentation broth allows for lower salt concentrations in the exiting fermentation broth, thus making the fermentation more efficient, which, as mentioned, translates into higher yields and lower operating and capital costs.

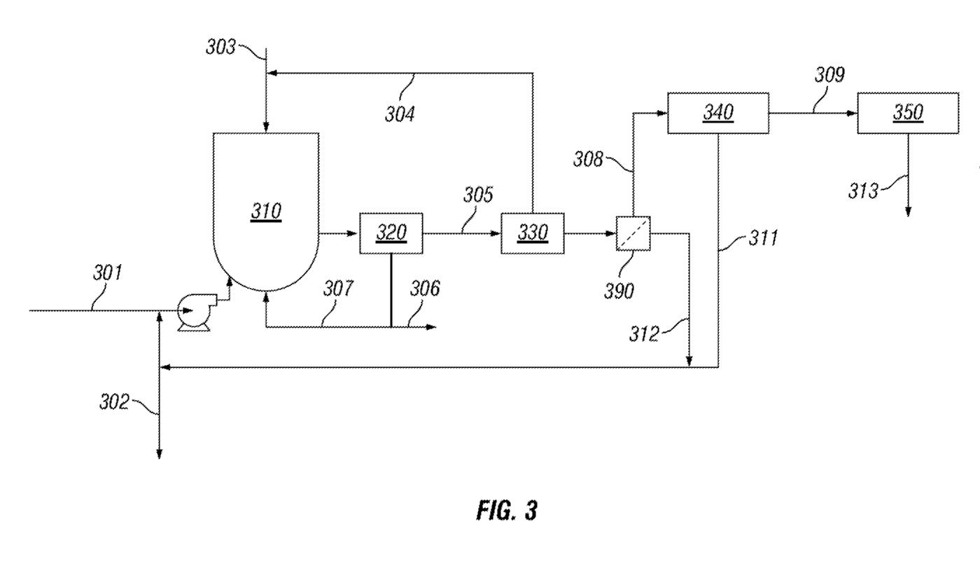

In an embodiment, as shown in FIG. 3, a low-solids concentration, low-product concentration (dilute), high-yield fermentation process is integrated with RO followed by evaporation to handle the dewatering of the produced fermentation broth. Fresh feedstock 303, which may be optionally pretreated if needed to enhance microbial conversion, is delivered to the fermentor 310. Fermentor 310 may be any type of fermentor. For example, a low-solid concentration fermentor/reactor is desired to take advantages of the low-product concentration because it simplifies the mixing mechanism and minimizes the energy required for mixing and pumping. Other additives as known to one skilled in the art may be added to the fermentation as needed (e.g., nutrients, buffering agent for pH control, methane inhibitors). To remove both liquid and solids, at least a portion of the fermentor content is sent to a liquid/solid separator 320 or to a series of liquid/solid separators. In various embodiments, these liquid/solid separators may include, but are not limited to, screens, or filters, or screw presses, or centrifuges, or combinations thereof.

To obtain a low LRT, a large quantity of slurry needs to be processed through the liquid/solid separator, and as a result a large quantity of solids separated in thk operation are recycled via line 307 and only a small amount is removed from the system as undigested residue (306). The liquid, on the other hand, is sent via line 305 to a pretreatment system 330, which may include, but it is not limited to, a high-speed centrifuge, or a flocculation/coagulation system, or a fine filtration system, or a microfiltration membrane system, or an ultrafiltration membrane system, or a nanofiltration membrane system or combinations thereof. From the pretreatment 330, some components, such as cells, proteins, fine suspended solids, etc (a.k.a. scum) are recovered and may be recycled back to the fermentation via line 304 or purged if desired. The pretreated liquid, on the other hand, is then sent to the RO system 390 where water is removed as permeate 312 and recycled to the fermentation. The now concentrated retentate, which is approximately at the switch concentration for optimal operation, is then sent via line 308 to an evaporation system 340, which may include, but it is not limited to, a multiple-effect evaporation system, or a multi-effect flash system, or a vapor-compression or vapor-recompression system, or combinations thereof. The concentrate from the evaporator 309 with the product, which is approximately at the desired final concentration, is then sent downstream for further processing 350. As an illustration, if the product from the fermentation is carboxylate salts, the downstream processing 350 may comprise crystallization of the salts, extraction of the acids, conversion into ketones, esters, alcohols and/or hydrocarbons. The final product 313, would then depend on the fermentation product and the downstream processing chosen, but it may be the carboxylate salts themselves, carboxylic acids, ketones, esters, alcohols and/or hydrocarbons. The condensate from the evaporation system 311, which is mostly pure water (although some volatiles like ammonia may be present), on the other hand, is recycled together with the RO permeate 312. Depending on the moisture content of the feedstock, it is possible that there might be some excess water that needs to be exported or make-up fresh water that needs to be added. Such adjustment is made via line 301 (fresh water in) and 302 (water out).

In some cases, gases leaving the fermentation need to be treated for odors and other gases, and, if they contain valuable products, such as hydrogen, they need to be recovered. Such recovery methods (separation of the hydrogen from the other gases) include pressure-swing absorption, or autotrophic bioconversion, where carbon dioxide and hydrogen are converted into product by autotrophic microorganisms.

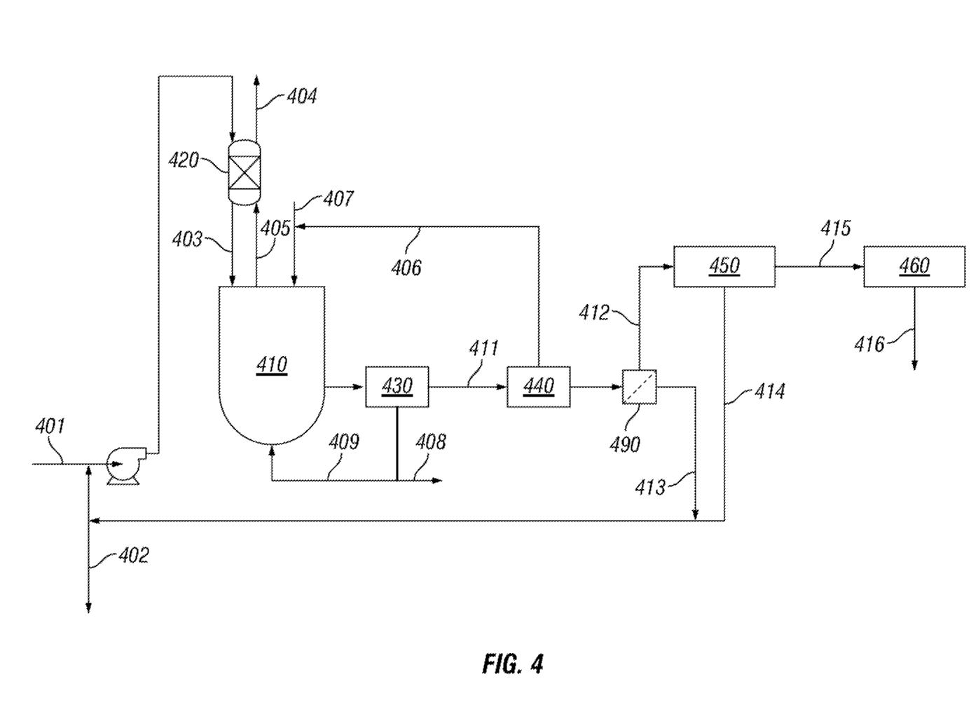

FIG. 4 illustrates a process wherein an autotrophic conversion is combined with the process as described in FIG. 3. FIG. 4 shows the same configuration as FIG. 3, but it also shows the fermentation gases and the water or part of the water coming from the evaporation and RO systems being directed to a bioreactor, where they are converted into product and exit the bioreactor as a dilute solution. This dilute solution may then be sent to the fermentation as the liquid input, or it may be sent directly to the RO system.

Fresh feedstock 407, which may be optionally pretreated if needed to enhance microbial conversion, is delivered to the fermentor 410. Bioreactor 420 for autotrophic conversion is coupled with fermentor 410 via line 403 (dilute product stream) and line 405 (fermentor gas). Bioreactor 420 has gas exhaust line 404.

To remove both liquid and solids, at least a portion of the fermentor content is sent to a liquid/solid separator 430 or to a series of liquid/solid separators. In various embodiments, these liquid/solid separators may include, but are not limited to, screens, or filters, or screw presses, or centrifuges, or combinations thereof.

To obtain a low LRT, a large quantity of slurry needs to be processed through the liquid/solid separator, and as a result a large quantity of solids separated in this operation are recycled via line 409 and only a small amount is removed from the system as undigested residue (408). The liquid, on the other hand, is sent via line 411 to a pretreatment system 440, which may include, but it is not limited to, a high-speed centrifuge, or a flocculation/coagulation system, or a fine filtration system, or a microfiltration membrane system, or an ultrafiltration membrane system, or a nanofiltration membrane system, or combinations thereof. From the pretreatment 440, some components, such as cells, proteins, fine suspended solids, etc (a.k.a. scum) are recovered and may be recycled back to the fermentation via line 406 or purged if desired.

The pretreated liquid, on the other hand, is then sent to the RO system 490 where water is removed as permeate 413 and recycled to the bioreactor/fermentor. The now concentrated retentate, which is approximately at the switch concentration for optimal operation, is then sent via line 412 to an evaporation system 450, which may include, but it is not limited to, a multiple-effect evaporation system, or a multi-effect flash system, or a vapor-compression or vapor-recompression system, or combinations thereof. The concentrate from the evaporator 415 with the product, which is approximately at the desired final concentration, is then sent downstream for further processing 460 to produce final products 416.

The condensate from the evaporation system 414, which is mostly pure water (although some volatiles like ammonia may be present), on the other hand, is recycled together with the RO permeate 413. Depending on the moisture content of the feedstock, it is possible that there might be some excess water that needs to be exported or make-up fresh water that needs to be added. Such adjustment is made via line 401 (fresh water in) and 402 (water out).

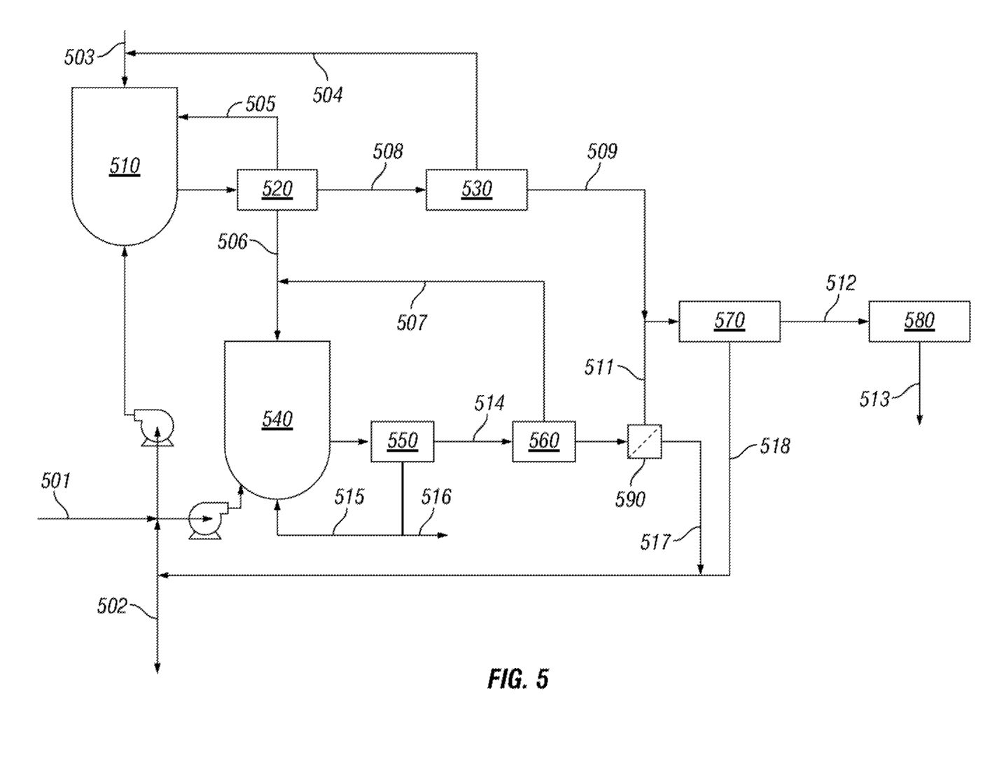

Integrated Fermentation. A low-product-concentration fermentation (termed “dilute fermentation” in this disclosure) is able to produce high yields with low solids concentrations inside the fermentor; higher acid concentrations may be attained at higher solids concentration but with low yields (termed “concentrated fermentation” in this disclosure). In an embodiment, an integration of these two fermentations (dilute fermentation and concentrated fermentation) gives more operational flexibility, e.g., reducing dewatering equipment needs and producing high yields. Such integration allows the poorly converted solids that exit the high-product-concentration fermentation (concentrated fermentation) to be highly converted in the low-product-concentration fermentation (dilute fermentation), and the low-product-concentration liquid that exits the low-product-concentration fermentation (dilute fermentation) to receive a boost in its product concentration if it is used as the inlet liquid entering the high-acid-concentration fermentation (concentrated fermentation). FIGS. 5 through 7 illustrate different configurations for this integrated process.

FIG. 5 shows the illustration in which the fresh feedstock 503 is fed to a high-solid concentration, high-product concentration, but low-yield fermentation 510. In an embodiment, this fermentation is performed in a fermentor or series of fermentors, which may be arranged and operated in a manner similar to what has been described by Holtzapple et al. A liquid/solid separator 520 is used to separate solids from the liquid, which may include, but is not limited to screens, or filters, or screw presses, or centrifuges, or a combination thereof. Because, typically, the LRT in this fermentor is high in comparison to the solid residence time, some of the fermentation liquid obtained from this liquid/solid separator is recycled to the fermentation via line 505. The fermentation liquid or broth, on the other hand, continues on via line 508 to a pretreatment system 530, which may include, but is not limited to, a high-speed centrifuge, or a flocculation/coagulation system, or a very fine filtration system, or a microfiltration membrane system, or an ultrafiltration membrane system, or a nanofiltration membrane system, or combinations thereof. The pretreatment 530 will remove any fine suspended solids, cells, large proteins etc (scum) and thus clarify the broth. The clarified fermentation liquid or broth that exits the pretreatment system (509) is at or above the switch fermentation, and it is sent directly to evaporation 570, while the scum 504 (including, e.g., cells, large proteins) may be recycled to the fermentation. The resulting poorly converted solids that are obtained from the liquid/solid separator from fermentation 510 are sent via line 506 to the fermentation process 540 that may occur at a lower solid concentration, low product concentration (dilute), but with a high yield. Generally speaking, low yield refers to a yield below 60% and high yield refers to a yield no less than 60%.

As with the process in FIG. 3, in FIG. 5 a liquid/solid separator 550 is used to separate the liquid from the solids, which may include, but is not limited to, screens, or filters, or screw presses, or centrifuges, or combinations thereof. Because the LRT is low, a lot of solids will need to be recycled (515), and only a small amount will exit the system as undigested residue (516). The low-product concentration (dilute) fermentation liquid or broth, on the other hand, continues on via line 514 to pretreatment 560, which may include, but it is not limited to, a high-speed centrifuge, or a flocculation/coagulation system, or a fine filtration system, or a microfiltration membrane system, or an ultrafiltration membrane system, or a nanofiltration membrane system, or combinations thereof. In the pretreatment system 560 components, such as cells, proteins, fine suspended solids, etc (scum) are recovered and may be recycled to the fermentation 540 via line 507. The resulting low-product concentration (dilute) clarified fermentation liquid or broth that exits this fermentation is treated as described in FIG. 3, where it is sent to an RO dewatering system 590, where the retentate is concentrated to the switch concentration and then sent, via line 511, to evaporation 570, which may include, but it is not limited to, a multiple-effect evaporation system, or a multi-effect flash system, or a vapor-compression or vapor-recompression system, or combinations thereof. The evaporation dewaters both the clarified liquid from fermentation 510 and the retentate from the RO system that processes the low-product concentration (dilute) clarified liquid from fermentation 540. The concentrate that is generated at the evaporation is sent downstream via line 512 for further processing 580. Such downstream processing includes, in the illustrative case of carboxylate salts (such as acetate), crystallization of the salts, extraction of the acids, conversion into ketones, esters, alcohols, hydrocarbons, etc. Final products exit the system via line 513. The permeate from the RO system 517 and the condensate from the evaporation system 518, which are mostly pure water, are recycled back to the fermentations, where part of this water is sent as liquid feed input to fermentation 510 and the remaining amount is sent as liquid input to fermentation 540. As with the process in FIG. 3, some water might need to be exported or made up depending on the moisture content of the incoming feedstock to fermentation 510. Such adjustments may be accomplished via line 501 (fresh water in) and line 502 (water out), depending on the moisture content of the incoming feedstock and the ability of the system to recycle condensate.

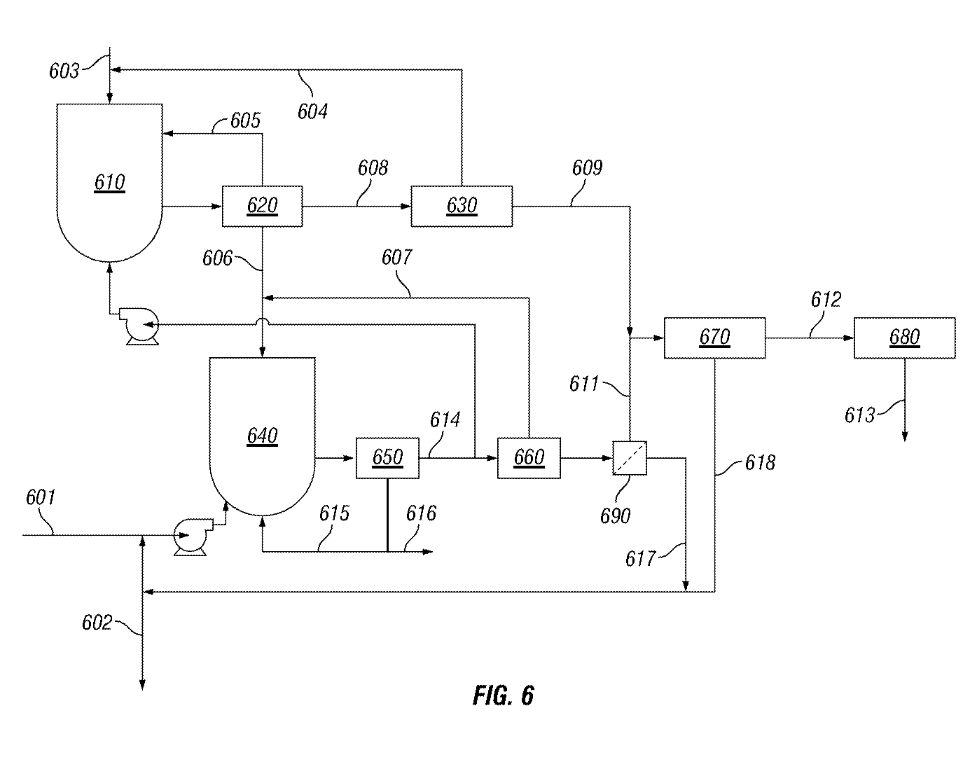

FIG. 6 shows a process very similar to the process illustrated in FIG. 5, except for the fact that the liquid input for fermentation 610 is obtained from part of the low-product-concentration fermentation liquid generated in fermentation 640. This low-product concentration (dilute) fermentation liquid may be obtained from the liquid stream right after the liquid/solid separator 650, thus keeping this portion of fermentation liquid from going through the pretreatment 660 and the RO system 690. The permeate 617 and condensate water streams 618 are recycled only to fermentation 640 as opposed to what is seen in FIG. 5, where part of these water streams are sent as liquid input to fermentation 510.

In FIG. 6, fresh feedstock 603 is fed to a high-solid concentration, high-product concentration, but low-yield fermentation 610. In an embodiment, this fermentation is performed in a fermentor or series of fermentors, which may be arranged and operated in a manner similar to what has been described by Holtzapple et al. A liquid/solid separator 620 is used to separate solids from the liquid, which may include, but is not limited to screens, or filters, or screw presses, or centrifuges, or a combination thereof. Because, typically, the LRT in this fermentor is high in comparison to the solid residence time, some of the fermentation liquid obtained from this liquid/solid separator is recycled to the fermentation via line 605. The fermentation liquid or broth, on the other hand, continues on via line 608 to a pretreatment system 630, which may include and is not limited to, a high-speed centrifuge, or a flocculation/coagulation system, or a very fine filtration system, or a microfiltration membrane system, or an ultrafiltration membrane system, or a nanofiltration membrane system or combinations thereof. The pretreatment 630 will remove any fine suspended solids, cells, large proteins etc (scum) and thus clarify the broth. The clarified fermentation liquid or broth that exits the pretreatment system (609) is at or above the switch fermentation, and it is sent directly to evaporation 670, while the scum 604 (including, e.g., cells, large proteins) may be recycled to the fermentation. The resulting poorly converted solids that are obtained from the liquid/solid separator from fermentation 610 are sent via line 606 to the fermentation process 640 that may occur at a lower solid concentration, low product concentration (dilute), but with a high yield.

In an embodiment, a liquid/solid separator 650 is used to separate the liquid from the solids for fermentation 640, which may include, but is not limited to, screens, or filters, screw presses, or centrifuges, or combinations thereof. Because the LRT is low, a lot of solids will need to be recycled (615), and only a small amount will exit the system as undigested residue (616). The low-product concentration (dilute) fermentation liquid or broth, on the other hand, continues on via line 614 to pretreatment 660, which may include, but it is not limited to, a high-speed centrifuge, or a flocculation/coagulation system, or a fine filtration system, or a microfiltration membrane system, or an ultrafiltration membrane system, or a nanofiltration membrane system, or combinations thereof. In the pretreatment system 660, components, such as cells, proteins, fine suspended solids, etc (scum) are recovered and may be recycled to the fermentation 640 via line 607. The resulting low-product concentration (dilute) clarified fermentation liquid or broth that exits this fermentation is treated as described in FIG. 3, where it is sent to an RO dewatering system 690, where the retentate is concentrated to the switch concentration and then sent, via line 611, to evaporation 670, which may include, but it is not limited to, a multiple-effect evaporation system, or a multi-effect flash system, or a vapor-compression or vapor-recompression system, or combinations thereof. The evaporation dewaters both the clarified liquid from fermentation 610 and the retentate from the RO system that processes the low-product concentration (dilute) clarified liquid from fermentation 640. The concentrate that is generated at the evaporation is sent downstream via line 612 for further processing 680. Such downstream processing includes, in the illustrative case of carboxylate salts (such as acetate), crystallization of the salts, extraction of the acids, conversion into ketones, esters, alcohols, hydrocarbons, etc. Final products exit the system via line 613. As with the process in FIG. 3, some water might need to be exported or made up depending on the moisture content of the incoming feedstock to fermentation 610. Such adjustments may be accomplished via line 601 (fresh water in) and line 602 (water out), depending on the moisture content of the incoming feedstock and the ability of the system to recycle condensate.

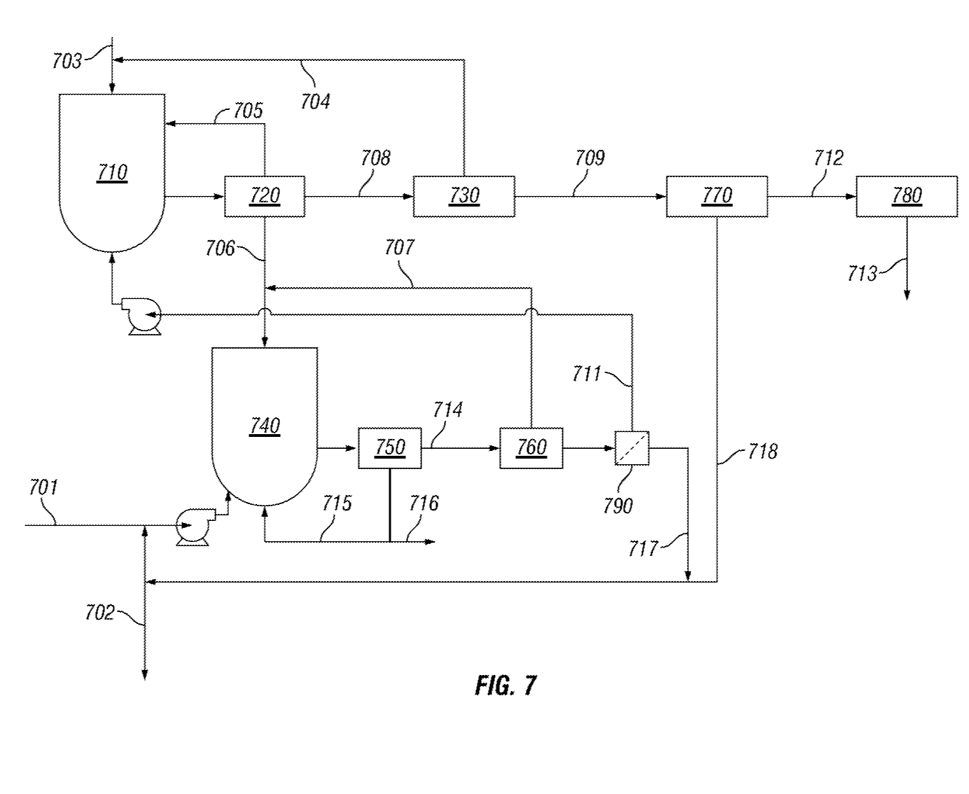

FIG. 7 also shows a process very similar to the process illustrated in FIG. 5, except for the fact that the liquid input for fermentation 710 is all the retentate 711 out of the RO membrane 790 that is processing the low-product-concentration liquid from fermentation 740. As opposed to the process in FIG. 5, the resulting retentate is not at the switch concentration, but at a lower concentration, and the concentration boost for this liquid stream is wholly experienced in fermentation 710, thus only the clarified fermentation liquid 709 that is exiting the pretreatment system 730 which should be at the same or higher concentration than the switch concentration, is sent to evaporation 770 while, again, all the retentate 711 is sent to fermentation 710. The advantage of this process is that the RO membrane would operate at lower pressures than the processes in FIG. 3 through 6 because of the lower osmotic pressure as the retentate concentration is lower than the switch concentration. In addition, the lower final concentration lowers fouling propensity and thus lowers maintenance needs. As with the process illustrated in FIG. 6, the permeate 717 and condensate 718 water streams are recycled only to fermentation 740 as opposed to what is seen in FIG. 5, where part of these water streams are sent as liquid input to fermentation 510.

In an embodiment as illustrated by FIG. 7, fresh feedstock 703 is fed to a high-solid concentration, high-product concentration, but low-yield fermentation 710. In an embodiment, this fermentation is performed in a fermentor or series of fermentors, which may be arranged and operated in a manner similar to what has been described by Holtzapple et al. A liquid/solid separator 720 is used to separate solids from the liquid, which may include, but is not limited to screens, or filters, or screw presses, or centrifuges or a combination thereof. Because, typically, the LRT in this fermentor is high in comparison to the solid residence time, some of the fermentation liquid obtained from this liquid/solid separator is recycled to the fermentation via line 705. The fermentation liquid or broth, on the other hand, continues on via line 708 to a pretreatment system 730, which may include and is not limited to, a high-speed centrifuge, or a flocculation/coagulation system, or a very fine filtration system, or a microfiltration membrane system, or an ultrafiltration membrane system, or a nanofiltration membrane system, or combinations thereof. The pretreatment 730 will remove any fine suspended solids, cells, large proteins etc (scum) and thus clarify the broth. The clarified fermentation liquid or broth that exits the pretreatment system (709) is at or above the switch fermentation, and it is sent directly to evaporation 770, which may include, but it is not limited to, a multiple-effect evaporation system, or a multi-effect flash system, or a vapor-compression or vapor-recompression system, or combinations thereof. The scum 704 (including, e.g., cells, large proteins) may be recycled to the fermentation. The concentrate that is generated at the evaporation system 770 is sent downstream via line 712 for further processing 780. Such downstream processing includes, in the illustrative case of carboxylate salts (such as acetate), crystallization of the salts, extraction of the acids, conversion into ketones, esters, alcohols, hydrocarbons, etc. Final products exit the system via line 713.

The resulting poorly converted solids that are obtained from the liquid/solid separator from fermentation 710 are sent via line 706 to the fermentation process 740 that may occur at a lower solid concentration, low product concentration (dilute), but with a high yield. In some embodiments, a liquid/solid separator 750 is used to separate the liquid from the solids for fermentation 740, which may include, but is not limited to, screens, or filters, or screw presses, or centrifuges, or combinations thereof. Because the LRT is low, a lot of solids will need to be recycled (715), and only a small amount will exit the system as undigested residue (716). The low-product concentration (dilute) fermentation liquid or broth, on the other hand, continues on via line 714 to pretreatment 760, which may include, but it is not limited to, a high-speed centrifuge, or a flocculation/coagulation system, or a fine filtration system, or a microfiltration membrane system, or an ultrafiltration membrane system, or a nanofiltration membrane system, or combinations thereof. In the pretreatment system 760, components, such as cells, proteins, fine suspended solids, etc (scum) are recovered and may be recycled to the fermentation 740 via line 707.

In some cases (not shown in FIG. 7), the permeate from the RO system 717 and the condensate from the evaporation system 718, which are mostly pure water, are recycled back to the fermentations, where part of this water is sent as liquid feed input to fermentation 710 and the remaining amount is sent as liquid input to fermentation 740. In some cases, the permeate 717 and condensate 718 water streams are recycled only to fermentation 740 as shown in FIG. 7. As with the process in FIG. 3, some water might need to be exported or made up depending on the moisture content of the incoming feedstock to fermentation 710. Such adjustments may be accomplished via line 701 (fresh water in) and line 702 (water out), depending on the moisture content of the incoming feedstock and the ability of the system to recycle condensate.

For the embodiments as illustrated by the configurations shown in FIGS. 3-7, as illustration, the following assumptions are made to simulate mass balances at different acid concentration scenarios in the low-solids-concentration, low-product-concentration, high-yield fermentation:
1) The fermentation is anaerobic digestion of biomass for producing carboxylic acids. In the case of carboxylic-acid production by a mixed culture of microorganisms, inhibition is not a serious limitation until the carboxylic acid salt concentration in the fermentation exceeds about 20 to 25 g/L of carboxylic acid equivalent; therefore, using the processes in FIGS. 3 and 5 through 7, the acid salt concentration is not expected to exceed more than 25 g/L in the dilute fermentation. The simulations are run at concentrations ranging from 5 g/L to 25 g/L of acid.
2) The moisture content in the feed is such that it balances with the water of hydrolysis consumed during microbial break down of biomass, the water lost with the undigested residue and the water produced from microbial growth so that no make-up water is needed and no extra water for export is produced. All the water is recycled within the process.
3) At all times in the fermentor or series of fermentors that compose the high-solids-concentration, high-product-concentration, low-yield fermentation (concentrated fermentation), 50 g/L of carboxylic acid is maintained and little acid produced in concentrated fermentation is transferred to dilute fermentation with the solids that exit the liquid-solid separation unit.
4) 50 g/L is the switch concentration, which, as mentioned before, represents the optimal concentration which RO should drive the concentration to before switching to evaporation to minimize costs.
5) The fermentation broth has a negligible amount of dissolved solids other than the acids or the salts of the acids.
6) The overall yield (g of carboxylic acid/g of biomass fed) is 50%, with 15% in the concentrated fermentation and 35% in the dilute fermentation. In the cases where there is no concentrated fermentation (e.g., the process shown in FIG. 3), the entire 50% yield is attained in the dilute fermentation.

Table 1 shows the results of such simulations. The combination of the two types of fermentations, i.e., concentrated fermentation (high-solids concentration, high-product concentration, low-yield fermentation) and dilute fermentation (low-solids concentration, low-product concentration/dilute, high-yield fermentation) gives the flexibility for optimization in view of capital and operating costs.

TABLE 1
Mass balance simulation of the different configuration in the integration of a high-
product-concentration fermentation and a low-product-concentration fermentation
with RO and evaporation as dewatering.

| | | Acid Concentration in Dilute Fermentation | | | | |
|---|---|---|---|---|---|---|
| | | 5 g/L | 10 g/L | 15 g/L | 20 g/L | 25 g/L |
| Process | % of H₂O in feed removed by RO | 90.4% | 80.8% | 71.1% | 61.2% | 51.3% |
| in FIG. 3 | % of FIG. 3 RO feed | 100.0% | 100.0% | 100.0% | 100.0% | 100.0% |
| | % of FIG. 3 permeate | 100.0% | 100.0% | 100.0% | 100.0% | 100.0% |
| | Final concentration out of RO | 50 g/L | 50 g/L | 50 g/L | 50 g/L | 50 g/L |
| Process | % of H₂O in feed removed by RO | 90.4% | 80.8% | 71.1% | 61.2% | 51.3% |
| in FIG. 5 | % of FIG. 3 RO feed | 70.0% | 70.0% | 70.0% | 70.0% | 70.0% |
| | % of FIG. 3 permeate | 70.0% | 70.0% | 70.0% | 70.0% | 70.0% |
| | Final concentration out of RO | 50 g/L | 50 g/L | 50 g/L | 50 g/L | 50 g/L |
| Process | % of H₂O in feed removed by RO | 86.4% | 72.6% | 58.7% | 44.6% | 30.4% |
| in FIG. 6 | % of FIG. 3 RO feed | 66.8% | 62.9% | 57.8% | 51.0% | 41.5% |
| | % of FIG. 3 permeate | 66.8% | 62.9% | 57.8% | 51.0% | 41.5% |
| | Final concentration out of RO | 50 g/L | 50 g/L | 50 g/L | 50 g/L | 50 g/L |
| Process | % of H₂O in feed removed by RO | 86.4% | 72.6% | 58.7% | 44.6% | 30.4% |
| in FIG. 7 | % of FIG. 3 RO feed | 70.0% | 70.0% | 70.0% | 70.0% | 70.0% |
| | % of FIG. 3 permeate | 66.8% | 62.9% | 57.8% | 51.0% | 41.5% |
| | Final concentration out of RO | 35.5 g/L | 35.5 g/L | 35.5 g/L | 35.5 g/L | 35.5 g/L |

The following are examples of when one system might be more advantageous over another:
1) If the membrane dewatering is the lowest cost component, then the embodiment in FIG. 3 is the most suitable because it does not need the concentrated fermentation, which represents a fermentor or a series of fermentors, to generate the desired concentration for evaporation and the desired yield.
2) If, on the other hand, the membrane dewatering unit is more expensive than having an extra fermentor (concentrated fermentation), then the embodiments in FIGS. 5 through 7 are more suitable.
3) If pure water is desired to enter the concentrate fermentation to facilitate attaining the desired yields in that fermentor or series of fermentors, then the embodiment in FIG. 5 is the most suitable, although it decreases the size of the dewatering unit in comparison to FIG. 3 only by 30%, no matter what concentration is attained in the dilute fermentation.
4) If the amount of liquid that is to be sent to the membrane system is to be decreased to have a smaller membrane system, then the embodiment in FIG. 6 is the most suitable, because it decreases the amount of feed going to the RO and thus the size of the membrane system as well. Such system gets smaller, in comparison to the embodiment, in FIG. 3 as the concentration in the dilute fermentation increases. Also, as mentioned before, the embodiment in FIG. 6 avoids the use of the pretreatment system in the liquid that is sent directly to the concentrated fermentation, thus decreasing the pretreatment system size.
5) If operating costs (i.e., pumping and maintenance costs) are a concern with the membrane system, then the embodiment in FIG. 7 is most suitable because it allows the membrane to operate at a final concentration that is lower than the required switch concentration, which is needed to make the evaporation system less costly than the membrane system. The lower concentration decreases the osmotic pressure, which decreases the need for high pumping pressures (lowering pumping costs) and it also minimizes fouling (lowering maintenance costs).

With the aid of this disclosure, one skilled in the art is able to design similar configurations (processes and systems) to meet various and specific needs. Therefore, all such configurations are considered to be within the scope of this disclosure.

### REFERENCES

1. van der Wielen, L. A. M.; Luyden, K. C. A. M. (1992) “Integrated product formation and recovery in fermentation,” Current Opinion in Biotechnology 3, 130-138.
2. Datta, R. (1981) “Acidogenic conversion of corn stover,” Biotechnology and Bioengineering, 23, 61-77.
3. Miller, T. L.; Wolin, M. J. (1995)“Bioconversion of cellulose to acetate with pure cultures of Ruminococcus albus and a hydrogen-using acetogen,” Applied Environmental Microbiology, 61(11), 3832-3835.
4. Gijzen, H. J.; Zwart, K. B.; Verhagen, F. J. M; Vogels, G. D. (1988) “High-rate two-phase process for the anaerobic degradation of cellulose, employing rumen microorganisms for an efficient acidogenesis,” Biotechnology and Bioengineering, 31, 418-425.
5. Hogsett, D. A. L. (1995) “Cellulose hydrolysis and fermentation by Clostridium thermocellum for the production of ethanol,” Ph.D. Dissertation, Darthmouth College, Hanover, N.H.
6. Blasig, J. D. (1991) “Volatile fatty acid fermentation of ALEX-treated newspaper and bagasse by rumen microorganisms,” Ph.D. Thesis, Texas A&M University, College Station, Tex.
7. Perry, R. H.; Green, D. W. (1997) “Perry's chemical engineers' handbook,” McGraw-Hill, Seventh Ed., pg. 22-48-22-61.
8. Perry, R. H.; Green, D. W. (1997) “Perry's chemical engineers' handbook,” McGraw-Hill, Seventh Ed., pg. 11-107-11-118.
9. Perry, R. H.; Green, D. W. (1997) “Perry's chemical engineers' handbook,” McGraw-Hill, Seventh Ed., pg. 13-1-13-108.
10. Holtzapple, M. T.; Davison, R. R.; Luettich; T. (1999) “Recovery of fermentation salts from dilute solutions,” U.S. Pat. No. 5,986,133
11. Perry, R. H.; Green, D. W. (1997) “Perry's chemical engineers' handbook,” McGraw-Hill, Seventh Ed., pg. 15-4-15-47
12. Ma, N.; Fan, X.; Quan, X.; Zhang, Y. (2009) “Ag—TiO2/HAP/Al2O3 bioceramic composite membrane: Fabrication, characterization and bactericidal activity,” Journal of Membrane science, 336, 109-117.
13. Egger, S.; Lehmann, R. P.; Height, M. J.; Loessner, M. J.; Schuppler, M. (2009) “Antimicrobial properties of novel silver-silica nanocomposite materials,” Applied Environmental Microbiology, 75(9), 2973-2976.
14. Kim, J.; Van der Bruggen, B. (2010) “The use of nanoparticles in polymeric and ceramic membranes structures: Review of manufacturing procedures and performance improvement for water treatment,” Environmental Pollution, 158, 2335-2349.
15. Giladi, M.; Porat, Y.; Blatt, A.; Wasserman, Y.; Kirson, E. D.; Dekel, E.; Palti, Y. (2008) “Microbial growth inhibition by alternating electric fields,” Antimicrobial agents and chemotheraphy, 52(10), 3517-3522
16. Solichien, M. S.; O'Brien, D.; Hammond, E. G.; Glatz, C. E. (1995) “Membrane-based extractive fermentation to produce propionic and acetic acids: Toxicity and mass transfer considerations,” Enzyme and Microbial Biotechnology, 17, 23-31.
17. Nuchnoi, P.; Izawa, I.; Nishio, Naomichi; Nagai, S. (1987) “Extractive acidogenic fermentation by a supported liquid membrane,” Journal of Fermentation Technology, 65(6), 699-702.
18. Busche, R. M. (1991)“Extractive fermentation of acetic acid,” Applied Biochemistry and Biotechnology, 28/29, 605-621.
19. Holtzapple; M.; Davison; R.; Loescher; M.; Ross; M. K. (1999) “Apparatus for producing organic acids,” U.S. Pat. No. 5,962,307.
20. Holtzapple; M.; Davison; R.; Loescher; M.; Ross; M. K. (1999) “Method and apparatus for producing organic acids,” U.S. Pat. No. 5,874,263.
21. Holtzapple; M.; Davison; R.; Granda, C.; Noyes, G.; Darlington, E. (2010) “System and method for processing biomass,” U.S. patent application Ser. No. 12/708,298.
22. Holtzapple; M.; Davison; R.; Granda, C.; Noyes, G.; Darlington, E. (2005) “System and method for processing biomass,” U.S. patent application Ser. No. 11/298,983.
23. Playne, M. J. (1980)“Volatile fatty acid production by anaerobic fermentation of ligno-cellulosic substrates,” In: M. Moo-Young (Ed.), Advances in Biotechnology, Vol II. Pergamon Press, New York, N.Y., pp. 85-90.

Various dimensions, sizes, quantities, volumes, rates, and other numerical parameters and numbers have been used for purposes of illustration and exemplification of the principles of the disclosure, and are not intended to limit the disclosure to the numerical parameters and numbers illustrated, described or otherwise stated herein. Likewise, unless specifically stated, the order of steps is not considered critical. The different teachings of the embodiments discussed below may be employed separately or in any suitable combination to produce desired results.

While preferred embodiments of the disclosure have been shown and described, modifications thereof can be made by one skilled in the art without departing from the spirit and teachings of the disclosure. The embodiments described herein are exemplary only, and are not intended to be limiting. Many variations and modifications of the disclosure disclosed herein are possible and are within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, and so forth). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, and the like.

Accordingly, the scope of protection is not limited by the description set out above but is only limited by the claims which follow, that scope including all equivalents of the subject matter of the claims. Each and every claim is incorporated into the specification as an embodiment of the present disclosure. Thus, the claims are a further description and are an addition to the preferred embodiments of the present disclosure. The disclosures of all patents, patent applications, and publications cited herein are hereby incorporated by reference, to the extent they provide exemplary, procedural or other details supplementary to those set forth herein.

What is claimed is:

1. A method comprising
a) fermenting biomass to produce a fermentation broth;
b) separating the fermentation broth into a liquid stream and a solid or slurry stream;
c) passing said liquid stream through a reverse osmosis membrane to obtain a permeate and a retentate; and
d) concentrating said retentate to produce a concentrate.

2. The method of claim 1 wherein Step d comprises evaporating said retentate to produce a condensate in addition to said concentrate.

3. The method of claim 1 wherein Step b comprises utilizing a separation system selected from the group consisting of a high-speed centrifuge, a flocculation/coagulation system, a fine filtration system, a microfiltration membrane system, an ultrafiltration membrane system, a nanofiltration membrane system, and combinations thereof.

4. The method of claim 1 wherein Step a further comprises producing a gas stream and recovering a gas product from said gas stream.

5. The method of claim 1 wherein Step a further comprises
fermenting said biomass in a fermentor to produce a gas stream; and
introducing said gas stream to a bioreactor wherein autotrophic conversion takes places and wherein said fermentor is configured to receive a product stream from said bioreactor.

6. The method of claim 5 wherein said gas stream comprises carbon dioxide and hydrogen.

7. The method of claim 1 wherein said reverse osmosis membrane comprises ceramic membranes.

8. The method of claim 1 wherein said reverse osmosis membrane comprises an anti-fouling mechanism.

9. The method of claim 8 wherein said anti-fouling mechanism comprises the application of electric fields.

10. The method of claim 1 wherein said reverse osmosis membrane is selected from the group consisting of a composite of ceramic membrane with silver, a composite of ceramic membrane with titanium, a composite of ceramic membrane with alumina; a nano-composite material containing silver, and a nano-composite material containing silica.

11. The method of claim 1 further comprising recycling said permeate and said condensate to Step a.

12. The method of claim 1 further comprising processing said concentrate to produce a product, wherein product is selected from the group consisting of salts, acids, ketones, esters, alcohols, and hydrocarbons.

13. A method comprising
a) fermenting biomass in a first fermentor to produce a first fermentation broth;
b) separating the first fermentation broth into a first liquid stream and a first solid or slurry stream;
c) introducing said first solid or slurry stream into a second fermentor to produce a second fermentation broth, wherein said second fermentor comprises a lower fermentation products concentration than the first fermentor;
d) separating the second fermentation broth into a second liquid stream and a second solid or slurry stream; and
e) passing said second liquid stream through a reverse osmosis membrane to obtain a permeate and a retentate.

14. The method of claim 13 further comprising concentrating said first liquid stream or concentrating said first liquid stream and said retentate.

15. The method of claim 14 wherein said concentrating comprises evaporation to produce a condensate and a concentrate.

16. The method of claim 15 further comprising recycling said permeate and said condensate to Step a.

17. The method of claim 15 further comprising recycling said permeate and said condensate to Step c.

18. The method of claim 13 wherein at least a portion of said second liquid stream is sent to Step a.

19. The method of claim 13 wherein said retentate is sent to Step a.

20. The method of claim 13 wherein Step b or Step d comprises utilizing a separation system selected from the group consisting of a high-speed centrifuge, a flocculation/coagulation system, a fine filtration system, a microfiltration membrane system, an ultrafiltration membrane system, a nanofiltration membrane system, and combinations thereof.
